(19) United States
(12) Patent Application Publication
(10) Pub. No.: US 2009/0312736 A1
(43) Pub. Date: Dec. 17, 2009

(54) HYBRID STRETCH EAR AND ABSORBENT ARTICLE INCLUDING THE SAME

(75) Inventors: Charles F. Schroer, JR., Camden, AR (US); Matthew J. Rinaldi, Englishtown, NJ (US); M. R. Macfarlan, Allentown, NJ (US)

Correspondence Address: DICKSTEIN SHAPIRO LLP, 1633 Broadway, NEW YORK, NY, 10019, US

(73) Assignee: Arquest, Inc., Cranbury, NJ (US)

(21) Appl. No.: 12/486,044

(22) Filed: Jun. 17, 2009

Related U.S. Application Data
(60) Provisional application No. 61/073,137, filed on Jun. 17, 2008.

(51) Int. Cl. A61F 13/56

(52) U.S. Cl. 604/385.24

(57) ABSTRACT

An absorbent article having a chassis with a front section, a rear section and opposed lateral side edges. A pair of hybrid stretch ears are attached to one of the front section and the rear section of the chassis. Each of the hybrid stretch ears include a non-elastic portion and an elastic portion, the non-elastic portion of each hybrid stretch ear being attached to one of the opposed lateral side-edges of the chassis. The non-elastic portion and the elastic portion of each hybrid stretch ear are sized and positioned relative to one another such that the elastic portion is positioned outside of respective leg opening areas defined by the opposed lateral side edges of the chassis when the absorbent article is secured to the torso of a wearer.

### CROSS REFERENCE TO RELATED APPLICATION

The present invention claims priority to U.S. provisional patent application No. 61/073,137, filed Jun. 17, 2008, the entire contents of which are incorporated herein by reference.

### FIELD OF THE INVENTION

The present invention relates to absorbent articles such as diapers, training pants, and the like. More specifically, the present invention relates to absorbent articles with a stretch ear (or side panel) having a non-elastic portion and an elastic portion that provides superior fit, comfort, ease of use, and/or manufacturing cost.

### BACKGROUND OF THE INVENTION

Absorbent articles such as disposable diapers, training pants, and the like are known for their major function of absorbing and containing body exudates. Such articles are thus intended to prevent the soiling, wetting, or other contamination of clothing or other articles, such as bedding, that come in contact with the wearer. In the case of disposable diapers, they typically include a liquid permeable topsheet, a liquid impermeable backsheet, an absorbent core positioned between the topsheet and the backsheet, and fastening tabs for securing the absorbent article about the wearer's waist.

These absorbent articles sometimes include elastic stretchable ears positioned in the waist portion of the absorbent article and assist in securing the absorbent article about the wearer's waist. The elastic stretchable ears provide a stretchable portion that encompasses all or most of the section of the elastic stretch ear that defines the leg opening of the absorbent article. Since the elastic stretch ear is typically formed by a stretchable film surrounded by non-woven material, these stretchable ears are not breathable and can thus cause discomfort to the wearer.

### SUMMARY OF THE INVENTION

The present invention provides a hybrid stretchable ear that, while being stretchable to assist in securing the absorbent article to a wearer's waist, also provides increased breathability to reduce discomfort. The hybrid stretch ear of the present invention includes a non-stretchable portion and a stretchable portion.

The stretchable portion is positioned relative to the non-stretchable portion such that, in use, the stretchable portion is located outside of the leg opening area of the absorbent article. The non-stretchable portion is preferably comprised of a non-woven or other breathable material which allows moisture to readily escape. The nature of the non-woven or breathable material also allows for some degree of expansion, thereby also allowing conformability around the leg of the wearer without the additional force from an elastic stretch ear made completely from a stretchable material. The use of the non-woven material also assists in reinforcing the stretch ear such that the amount of necking down of the material is reduced as the ear is stretched.

### BRIEF DESCRIPTION OF THE DRAWINGS

The figures are for illustration purposes only and are not necessarily drawn to scale. The invention itself, however, may best be understood by reference to the detailed description which follows when taken in conjunction with the accompanying drawings in which:

FIG. 1 is a top plan view of an absorbent article having the hybrid stretch ear of the present invention as viewed from the outer side that contacts the clothing of a wearer;

FIG. 2 is a top plan view of the absorbent article of FIG. 1 as viewed from the inner side that contacts the body of the wearer; and

FIG. 3 is a perspective view of the absorbent article of FIG. 1 secured around the torso of the wearer.

### DETAILED DESCRIPTION OF THE INVENTION

The present invention will next be illustrated with reference to the figures. Such figures are intended to be illustrative rather than limiting and are included herewith to facilitate the explanation of exemplary features of embodiments of the present invention. Unless otherwise noted, the figures are not to scale, and are not intended to serve as engineering drawings.

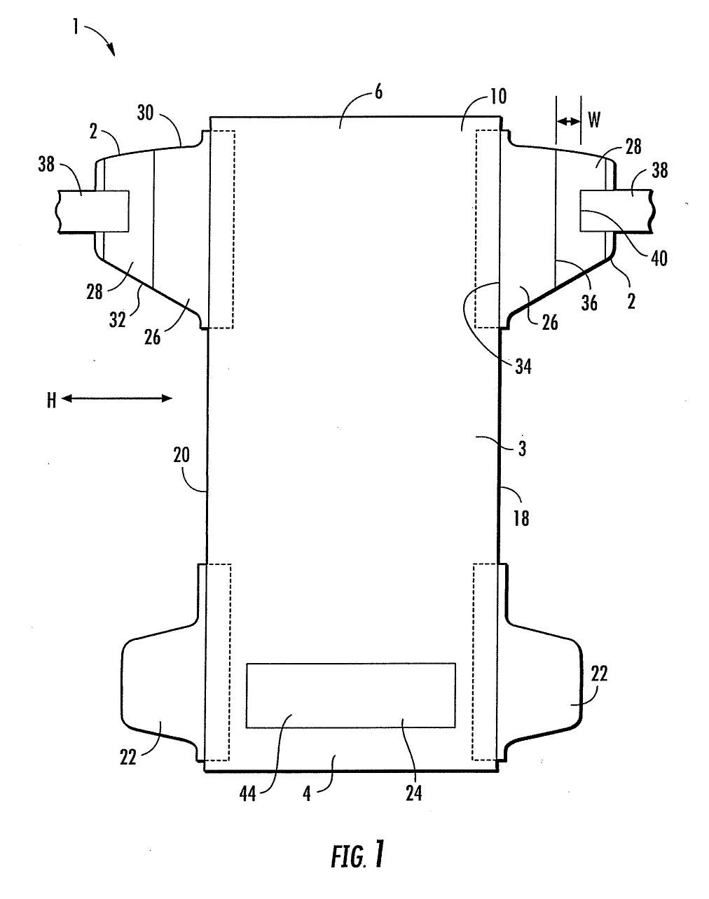

Referring now to the drawings, FIGS. 1-3 show an absorbent article 1 including a pair of hybrid stretch ears 2 of the present invention. The absorbent article 1 has a chassis 3 with a front section 4 and a rear section 6. As is known to one of skill in the art, the chassis 3 of a typical absorbent article includes a liquid permeable topsheet 8, a liquid impermeable backsheet 10, and an absorbent core 12 positioned between the topsheet 8 and the backsheet 10. The backsheet 10 can be composed of a single liquid impermeable material layer, or an outer liquid permeable material layer and an inner liquid impermeable material layer between the outer layer and the absorbent core 12. The absorbent article can also include barrier cuffs 14, 16 and elastic leg cuffs 15, 17, each located between the absorbent core and lateral side edges 18, 20 of the chassis 3 to assist in the containment of exudates within the absorbent article. Optional grasping tabs 22 are attached to the front section 4 of the chassis 3 adjacent a landing zone 24 and opposite the hybrid stretch ears 2.

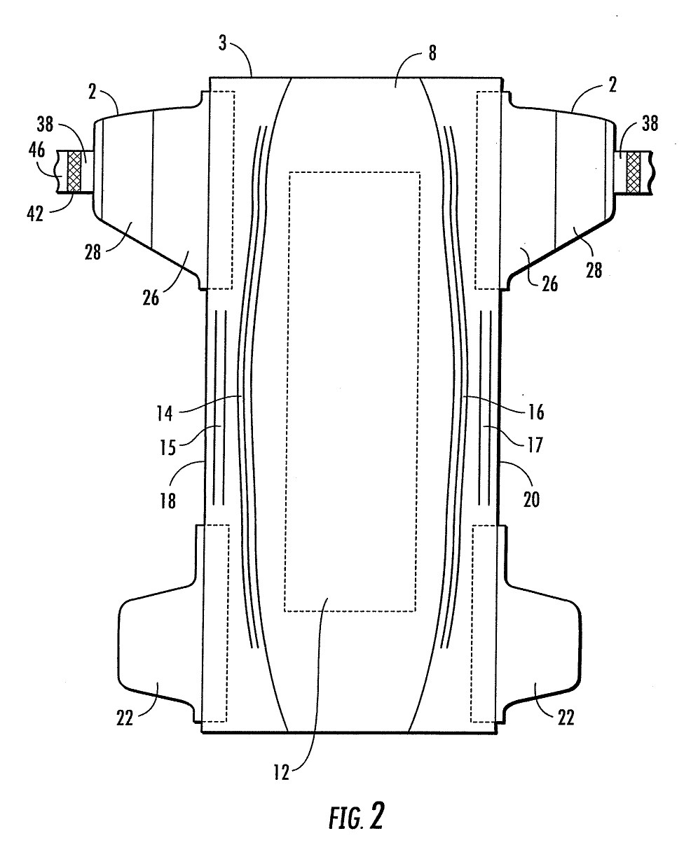

Preferably, the pair of hybrid stretch ears 2 are attached to the rear section 6 of the chassis 3. Each of the hybrid stretch ears 2 includes a non-elastic portion 26 and an elastic portion 28. The non-elastic portion 26 and the elastic portion 28 have a top edge 30 and a bottom edge 32 that are preferably asymmetrical to each other with respect to a horizontal axis H. The non-elastic portion 26 is attached to a lateral side edge 18, 20 of the chassis 3 along a first connecting edge 34, and coupled to the elastic portion 28 along a second connecting edge 36 distal from the first connecting edge 34 relative to the lateral side edge 18, 20. Preferably, and as shown in FIGS. 1 and 2, the hybrid stretch ears 2 are attached to the chassis 3 between the topsheet 8 and the backsheet 10.

A fastener 38 is attached to the elastic portion 28 of the hybrid stretch ear 2, or at an edge of the elastic portion 28 most distal from the lateral side edge 18, 20 of the chassis 3. Attachment of these portions to each other may be by an adhesive bond, an ultrasonic bond, a heat seal, stitching or a combination thereof or any other known coupling mechanism. In addition, the non-elastic portion 26 and the elastic portion 28 may be formed as an integral laminated structure comprising a non-elastic material layer and an elastic material layer. A second non-elastic material layer may also be provided to form a three-layer structure with the elastic material layer positioned between the two non-elastic material layers.

Preferably, the hybrid stretch ear 2 has a stretchable material adhesively laminated between two opposed layers of a spun-laced non-woven material. The stretchable material is preferably positioned 5 mm inward from the edge of the hybrid stretch ear 2 most distal to the lateral side edge 18, 20 of the chassis 3, and has a width of 40 mm. The non-elastic portion preferably is 55 mm in width. Other widths may be used for the elastic and non-elastic portions depending on the requirements of the particular absorbent article such as size and intended use, to name a few. For example, a smaller size ear for a smaller sized absorbent article could use a stretchable material having a width of 35 mm along with a non-elastic portion having a width of 36 mm.

While the stretchable material is preferably 40 mm in width, it is preferred that the fastener 38 be attached to the elastic portion 28 at a location such that the hybrid stretch ear 2 has an operable stretchable zone approximately 25 mm in width W (measured from the second connecting edge 36 to a bond 40 proximal thereto that attaches the fastener 38 to the ear 2) when the hybrid stretch ear is in a relaxed, unstretched state. The use of an operable stretchable zone less than the full width of the stretchable material provides a controlled amount of stretch to the stretch ear. As will be readily apparent, a smaller operable stretchable zone can be incorporated into smaller sized absorbent articles, for example, a 20 mm operable stretchable zone could be used on a stretch ear incorporating a 35 mm width stretchable material.

The non-elastic material of the hybrid stretch ear 2 may be selected from a variety of textile-like fabrics. Suitable fabrics include non-woven materials that are pervious to liquid, and are soft and pliable. Preferred non-woven materials include spun-laced polypropylene, spun-bonded polypropylene, spun-bonded polyethylene, spun-laced polyethylene, carded thermally bonded webs of staple fibers, preferably polypropylene shape or sheath/core bi-component fibers having a core of polyester or polypropylene and a sheath of polyethylene. The elastic material may be an elastic film or foam made of synthetic or natural rubber, or other elastomeric materials.

The fastener 38 may be any known releasable fastener, including adhesives, co-adhesives, tapes, buttons, hook and loop materials, and other fastening mechanisms used in the art, with the complementary fastener as a receptive area on what is commonly called the landing zone 24 of the absorbent article 1. The fastener 38 is preferably comprised of the hook portion 42 of a hook and loop fastening system. The loop portion 44 of the hook and loop fastening system is located on the landing zone 24 of the chassis 3 opposite the hybrid stretch ears 2, and is configured to engage with the hook portion 42. The fastener 38 is preferably re-fastenable and includes a tab component 46 that allows easy detachment of the hook portion 42 from the loop portion 44 , or, in general, the fastener 38 from the landing zone 24 of the absorbent article 1. The fastener 38 may comprise an inelastic material as a base material and may further include a finger tab at one end as the tab component 46.

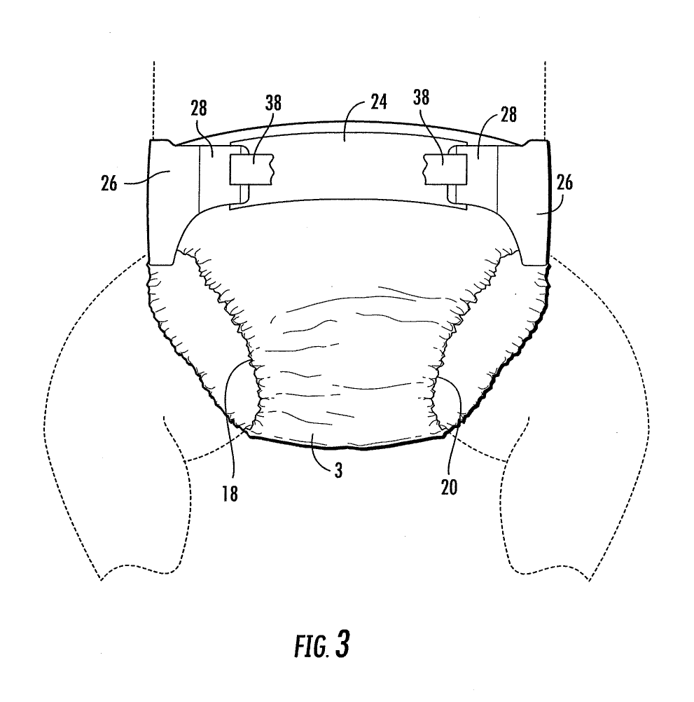

FIG. 3 shows the absorbent article 1 secured around the torso of a wearer with the elastic portion 28 of the hybrid stretch ear 2 in a stretched state. As shown in FIG. 3, the non-elastic portion 26 and the elastic portion 28 are sized and positioned relative to one another such that the elastic portion 28 is positioned outside of the leg opening area defined by the lateral side edges 18, 20 of the chassis 3 when the absorbent article is secured about a wearer's torso. In other words, the leg opening area of the absorbent article of the present invention can be partially defined by the non-elastic portion 26 of the hybrid stretch ear 2 and the lateral side edge 18, 20 of the chassis 3. With this configuration, the breathable material of the non-elastic portion 26 readily allows for the release of moisture that may be located between the leg of the wearer and the hybrid stretch ear 2. The nature of the non-woven or breathable material of the non-elastic portion 26 also allows for some degree of expansion, thereby also allowing conformability around the leg of the wearer without the additional force created by a stretch ear made completely from a stretchable material.

Alternatively, the leg opening area could be at least partially defined by the elastic portion 28 of the hybrid stretch ear 2 in a stretched state. This configuration of the hybrid stretch ear is preferred when the hybrid stretch ear is of a smaller overall size so as to reduce the amount of material used, and thus reduce the overall cost. Even with the use of a smaller sized hybrid stretch ear so that the elastic portion forms at least a part of the leg opening area, the operable stretchable zone is preferably 25 mm or less.

In addition to the above benefits, the hybrid stretch ear of the present invention reduces the cost of materials required for manufacturing because less elastic material is required to produce the hybrid stretch ear.

Although the hybrid stretch ear of the present invention has been shown and described in relation to a certain overall shape, other shapes are contemplated and include square, rectangular, triangular, polygonal, parallelogram, trapezoidal, elliptical, or any other geometric shapes. In particular, the hybrid stretch ear of the present invention can be formed with a linear top edge, a convex top edge, a concave top edge, a linear bottom edge, a convex bottom edge, a concave bottom edge, or any combination of top edge and bottom edge shapes.

Although the present invention has been particularly described in conjunction with specific embodiments, it is evident that certain alternatives, modifications, and variations will be apparent to those skilled in the art. Therefore, the present invention should not be limited by the specific disclosure herein, but only by the appended claims.

What is claimed is:

1. An absorbent article comprising:
a chassis having a front section, a rear section, and opposed lateral side edges;
a pair of hybrid stretch ears attached to one of the front section and the rear section of the chassis, each of the hybrid stretch ears including a non-elastic portion and an elastic portion, the non-elastic portion of each hybrid stretch ear being attached to one of the opposed lateral side-edges of the chassis; and
a respective fastener attached to each hybrid stretch ear of the pair of hybrid stretch ears.

2. The absorbent article of claim 1, wherein the chassis includes a liquid permeable topsheet, a liquid impermeable backsheet, and an absorbent core positioned between the topsheet and the backsheet.

3. The absorbent article of claim 1, wherein the respective fastener is attached to the elastic portion of each hybrid stretch ear of the pair of hybrid stretch ears.

4. The absorbent article of claim 1, wherein the non-elastic portion and the elastic portion have a top edge and a bottom edge that are asymmetrical to each other with respect to a horizontal axis.

5. The absorbent article of claim 1, wherein the non-elastic portion and the elastic portion are an integral laminated structure.

6. The absorbent article of claim 1, wherein the pair of hybrid stretch ears each have an operable stretch zone of approximately 25 mm or less in width in a relaxed state.

7. The absorbent article of claim 1, wherein the opposed leg opening areas are at least partially defined by the non-elastic portion of the hybrid stretch ears.

8. An absorbent article comprising:
a chassis having a front section, a rear section, and opposed lateral side edges;
a pair of hybrid stretch ears attached to one of the front section and the rear section of the chassis, each of the hybrid stretch ears including a non-elastic portion and an elastic portion, the non-elastic portion of each hybrid stretch ear being attached to one of the opposed lateral side-edges of the chassis; and
a respective fastener attached to each hybrid stretch ear of the pair of hybrid stretch ears,
wherein the non-elastic portion and the elastic portion of each hybrid stretch ear are sized and positioned relative to one another such that the elastic portion is positioned outside of respective leg opening areas defined by the opposed lateral side edges of the chassis when the absorbent article is secured to the torso of a wearer.

9. The absorbent article of claim 8, wherein the chassis includes a liquid permeable topsheet, a liquid impermeable backsheet, and an absorbent core positioned between the topsheet and the backsheet.

10. The absorbent article of claim 8, wherein the respective fastener is attached to the elastic portion of each hybrid stretch ear of the pair of hybrid stretch ears.

11. The absorbent article of claim 8, wherein the non-elastic portion and the elastic portion have a top edge and a bottom edge that are asymmetrical to each other with respect to a horizontal axis.

12. The absorbent article of claim 8, wherein the non-elastic portion and the elastic portion are an integral laminated structure.

13. The absorbent article of claim 8, wherein the pair of hybrid stretch ears each have an operable stretch zone approximately 25 mm in width in a relaxed state.

14. The absorbent article of claim 8, wherein the opposed leg opening areas are partially defined by the non-elastic portion of the hybrid stretch ears.

15. An absorbent article comprising:
a chassis having a front section, a rear section, and opposed lateral side edges;
a pair of hybrid stretch ears attached to one of the front section and the rear section of the chassis, each of the hybrid stretch ears including a non-elastic portion and an elastic portion, the non-elastic portion of each hybrid stretch ear being attached to one of the opposed lateral side-edges of the chassis; and
a respective fastener attached to each hybrid stretch ear of the pair of hybrid stretch ears,
wherein the non-elastic portion and the elastic portion of each hybrid stretch ear are sized and positioned relative to one another such that the elastic portion is positioned outside of respective leg opening areas defined by the opposed lateral side edges of the chassis when the absorbent article is secured to the torso of a wearer, the pair of hybrid stretch ears each have an operable stretch zone approximately 25 mm or less in width in a relaxed state, and the opposed leg opening areas are at least partially defined by the non-elastic portion of the hybrid stretch ears.

16. The absorbent article of claim 15, wherein the chassis includes a liquid permeable topsheet, a liquid impermeable backsheet, and an absorbent core positioned between the topsheet and the backsheet.

17. The absorbent article of claim 15, wherein the respective fastener is attached to the elastic portion of each hybrid stretch ear of the pair of hybrid stretch ears.

18. The absorbent article of claim 15, wherein the non-elastic portion and the elastic portion have a top edge and a bottom edge that are asymmetrical to each other with respect to a horizontal axis.

19. The absorbent article of claim 15, wherein the non-elastic portion and the elastic portion are an integral laminated structure.
